(19) United States
(12) Patent Application Publication
(10) Pub. No.: US 2003/0232068 A1
(43) Pub. Date: Dec. 18, 2003

(54) Food product having increased bile acid binding capacity

(76) Inventors: Daniel J. Lewandowski, Bloomington, MN (US); Ravindranath Menon, Rogers, MN (US); David Plank, Taylors Falls, MN (US)

Correspondence Address: GENERAL MILLS, INC., P.O. BOX 1113, MINNEAPOLIS, MN, 55440, US

(21) Appl. No.: 10/172,471

(22) Filed: Jun. 14, 2002

(51) Int. Cl. A61K 31/724; A61K 31/715; A61K 31/56; A61K 47/00

(52) U.S. Cl. 424/439; 514/58; 514/54; 514/171

(57) ABSTRACT

The present invention is related to a novel component for use in a consumer food product. More specifically, the ingredient or component, provided either alone or acting synergistically with other select ingredients, is part of an ingestible food product intended for human or animal consumption that provides a health benefit. The food product provides beneficial hypocholesterolemic activity through increased bile acid binding activity through the use of cyclodextrins alone or in combination with dietary fibers and/or sterols while simultaneously delivering a food product which is not adversely affected by its inclusion, either in taste or texture or in any undesirable side effects.

### CROSS-REFERENCES TO RELATED APPLICATIONS

None.

### BACKGROUND OF THE INVENTION

The present invention is related to a novel component for use in a consumer food product. More specifically, the ingredient or component, provided either alone or acting synergistically with other select ingredients is part of an ingestible food product intended for human or animal consumption that provides a health benefit. The food product provides beneficial hypocholesterolemic activity through increased bile acid binding activity while simultaneously delivering a food product which is not adversely affected by its inclusion, either in taste or texture or in any undesirable side effects.

There is a large amount of information in circulation today concerning elevated cholesterol levels and the health consequences due to that condition. In an effort to combat this result, a number of pharmaceutical applications, dietary supplements and other solutions relating to the treatment of high cholesterol levels have been previously introduced. However, regrettably, many of these products have an unpleasant mouth feel, that is they can feel slimy, have a displeasing taste or result in undesirable side effects which diminishes their overall value to the intended end user.

In addition, there also appears to be a growing disdain against ingesting some sort of dietary supplement, pharmaceutical treatment or other product to attain some perceived beneficial effect from such products. This may be due to a growing reliance on pills or tablets to sustain or maintain our health. Such reliance on supplements may also surprisingly contribute to malnutrition as other valuable vitamins and minerals can be omitted or overlooked when too much focus is diverted to certain items. Moreover, certain supplements may actually remove valuable macronutrients and micronutrients from the system. Individuals may also be concerned with potential risks and side effects associated with certain medications, treatments or supplements. In fact, dietary restrictions and other health concerns may preclude certain portions of the population from even consuming such products. As such, there remains a continuing interest in developing good tasting, well balanced, food products that contribute to a well balanced diet as well as provide a vehicle by which to deliver the benefit of cholesterol reduction in a palatable and efficient manner to meet the changing needs of the population.

Cholesterol in humans is known to come from primarily two sources, the body's own production of cholesterol (endogenous) and dietary cholesterol (exogenous). Lipoproteins contain specific proteins and varying amounts of cholesterol, triglycerides and phospholipids.

Bile acids are synthesized from cholesterol in the liver and then secreted into the intestines. Reducing the level of bile acid reabsorption facilitates the maintenance of a healthy cholesterol level. One method for reducing bile acid reabsorption is achieved by increasing the gut viscosity. Alternatively, a non-digestible dietary component which binds bile acids secreted in the proximal jejunum will reduce bile acid reabsorption in the lower intestines (distal ileum).

There are three major classes of lipoproteins and they include very low density lipoproteins (“VLDL”), low density lipoproteins (“LDL”) and high density lipoproteins (“HDL”). The LDLs are believed to carry about 60-70% of the serum cholesterol present in an average adult. The HDLs carry around 20-30% of serum cholesterol with the VLDL having around 1-10% of the cholesterol in the serum. To calculate the level of non-HDL cholesterol present (find the level of LDL or VLDL levels), which indicates risk; the HDL is subtracted from the total cholesterol value.

Typically, the average person consumes between 350-400 milligrams of cholesterol daily, while the recommended intake is around 300 milligrams. Increased dietary cholesterol consumption, especially in conjunction with a diet high in saturated fat intake, can result in elevated serum cholesterol. Having an elevated serum cholesterol level is a well-established risk factor for heart disease and therefore there is a need to mitigate the undesired effects of cholesterol accumulation. High cholesterol levels are generally considered to be those total cholesterol levels at 200 milligrams and above or LDL cholesterol levels at 130 milligrams and above. By lowering the total system LDL cholesterol level, it is believed that certain health risks, such as coronary disease and possibly some cancers, that are typically associated with high cholesterol levels, can be reduced by not an insignificant amount.

Numerous studies relating to modifying the intestinal metabolism of lipids have been done to illustrate that such effects can reduce a high cholesterol level. Hampering the absorption of triglycerides, cholesterol or bile acids or a combination of these items results in a lowering of cholesterol levels in the serum.

Soluble dietary fiber is known to be a safe ingredient due to its long history in food supply. Soluble fiber typically remains undigested, except by colonic microflora present in the lower intestines. Soluble dietary fiber is believed to have a beneficial effect in the reduction of high serum cholesterol levels and reducing the risk associated with such elevated levels. In addition, soluble dietary fiber can have the additional beneficial effect of reduced constipation and improved regularity. However, too much fiber in the diet can create undesirable gastrointestinal side effects such as flatulence, diarrhea, and abdominal cramps, etc. leading consumers to stay away from food products that contain too much dietary fiber, regardless of any associated health benefits. While some consumers may not completely avoid such products, they also do not typically regularly use such products due to the problems enumerated above or alternatively, or in combination due to the unpleasant taste of such products. This illustrates some of the problems with prior solutions that were aimed at providing high fiber diets directed at lowering cholesterol levels, and highlights the need to create a more balanced solution that fits not only within more normal dietary patterns but also meets consumer demand for better tasting, healthy products.

There are a number of other products purporting to have cholesterol-lowering properties available in the market today. One such product offering or solution is described in U.S. Pat. No. 6,136,349 which relates to a food product, food additive or the like that may be fortified with a select group of minerals, such as calcium, magnesium or potassium which when combined with conventional sterols and/or stanols increases the effect of the sterols and/or stanols in lowering cholesterol levels than with just sterols an/or stanols alone. However, significantly increasing only certain nutrients and minerals while ignoring others can result in over consumption or under consumption of essential nutrients because some nutrients are present in very high concentrations while other nutrients are present in very low concentrations. This creates a nutritionally unbalanced situation causing the consumer to either procure the missing macro and/or micronutrients through other food sources or omit them from their diet altogether. In addition to not receiving the DV (Daily Value) of certain nutrients, this may force the consumer into an over compensation mode causing the consumer to ingest more food than is actually necessary thereby defeating the purpose of such cholesterol-lowering foods, and potentially create other problems such as weight gain.

Another possible solution is described in U.S. Pat. No. 6,174,560, which relates to a food composition for lowering low-density cholesterol levels (LDL) and focuses on the use of at least one stanol fatty acid ester in combination with a nutritional substance. The applicants of U.S. Pat. No. 6,174,560 however indicates that increasing the amount of fiber to reduce serum cholesterol levels has been of a limited effect and citing that fiber that is delivered in therapeutically effective doses, such as with pharmaceutical applications, can cause extreme abdominal discomfort. This provides another singular example of a particular element or component being relied upon for a health effect but still ignoring the combined beneficial effects of the present invention as well as the ability to deliver the food product in an acceptable manner.

U.S. Pat. No. 5,244,887 describes the use of stanols as food additives to reduce cholesterol absorption. In the preparation of the additives, sitostanol is dissolved with an edible solubilizing agent such as triglyceride, an antioxidant such as tocopherol, and a dispersant such as lecithin, polysorbate 80, or sodium lauryl sulfate. However, no data is provided in the selection of the most effective components and their amounts or specific methods of preparation. Effectiveness in reducing cholesterol absorption was also not determined. The preferred embodiment consisted of 25% by weight stanols in vegetable oil, but the solubility of sterols in oil is only 2%.

Another difficulty with many of the prior art solutions, regardless of whether they are successful in lowering cholesterol levels or not, is simply a matter of the cost of the ingredients or components which are needed to achieve the desired benefit. Only a very small segment of the population may be willing to pay eight or even ten dollars for a box of cereal or a loaf of bread, despite the benefit associated with it. In addition even if consumers purchase such a product initially, the high cost is likely to be more of a disincentive to purchase the product in the future, when compared with the incentive of the health benefit associated with the product.

Another problem associated with such prior art food problems is that the consumer may be forced to eat several servings of the food product in order to attain the benefit of cholesterol reduction. This further complicates the delivery of the health benefit to the consumer in that a consumer may not want to eat a half a loaf of bread or consume three or more bowls of cereal at a meal. Moreover, over consumption can lead to other problems such as weight gain.

A focus of the present invention relates to a novel use of cyclodextrins, either individually or synergistically, in connection with other ingredients or components to enhance the hypocholesterolemic benefit. It has been found that when cyclodextrins are used with other ingredients or components the level of such other health giving components (such as dietary fiber) may be reduced. Such an unanticipated benefit permits the manufacturer to more efficiently manufacture products, by removing high cost ingredients, which meet the budgetary restrictions of consumers.

Cyclodextrins comprise a doughnut shaped or cyclical structure composed of between six to eight alpha-D-glucose units having a hydrophilic exterior (hydrophilic OH groups on the exterior rim) and a hydrophobic interior (electron dense hydrogen and oxygen atoms). Cyclodextrins are generally water soluble, free flowing crystalline powders that are substantially if not completely odorless and white in color.

Heretofore, starches such as cyclodextrins have not been employed or known for their hypocholesterolemic activity in humans. Cyclodextrins have been used principally for the encapsulation of insoluble compounds on a molecular basis in order to enhance stability, reduce volatility and alter solubility as well as to increase shelf life of certain products. Such prior uses of cyclodextrins have been limited to flavor carriers and protection of sensitive substances against thermal decomposition, oxidation and degradation. In addition, more recently, cyclodextrins have also been used to remove fatty acids and cholesterol from animal fats and to remove cholesterol and cholesterol esters from egg yolks.

Another potential solution to the high cholesterol problem teaches the treatment of the foodstuffs themselves with cyclodextrins rather than the consumer. U.S. Pat. Nos. 5,498,437, 5,342,633 and 5,063,077 discuss various processes for the removal of cholesterol and cholesterol esters from egg yolks, meat, animal fats, etc. It is thought that by reducing the level of cholesterol in such foodstuffs that overall levels of cholesterol may be reduced in consumers. However, processing steps to such foodstuffs increases the cost of delivering such products to market.

Another similar but apparently unrelated reference, which deals with removal of cholesterol from foodstuffs, is U.S. Pat. No. 5,232,725. This reference discusses a process for reducing cholesterol and free fatty acids in an animal fat and the material obtained from that process through the use of cyclodextrins. U.S. Pat. No. 5,223,295 also discusses the use of cyclodextrin to remove steroid based compounds from foodstuffs, particularly egg yolks. However, these patents suffer from the same drawbacks as those referenced above, in that the processing steps required to achieve the result adds another layer to delivering product to the market, causing delay and adding cost.

A further drawback associated with such prior solutions of treating the foodstuff as opposed to treating the consumer, is that the consumer may then be tempted to eat more of the product sensing that the cholesterol level of the product has been reduced, that is the consumer may have a four egg omelet instead of two, thus ingesting the same level of cholesterol as before, thereby defeating the overall purpose of the solution.

PCT Publications WO 99/59421 and WO 99/63841 disclose the use of phytosterols as a pharmaceutical agent or as an addition to certain foodstuffs for lowering cholesterol. The publication discusses that greater effectiveness of the phytosterols can be achieved when using a specified delivery vehicle such as a complexation with cyclodextrins. This represents little more than using cyclodextrins for a purpose that they are already known for, as a carrier for sensitive ingredients.

Another reference that teaches the use of beta-cyclodextrin as a carrier or delivery vehicle is U.S. Pat. No. 4,978,532. In this reference, dehydroepiandrosterone (DHEA) is delivered to the patient via a treatment patch. Beta-cyclodextrin is selected from a group of “permeation enhancers” to facilitate the delivery of the DHEA dose to the patient.

U.S. Pat. No. 5,624,940 the use of various complexes which include cyclodextrins for reducing bone loss and serum cholesterol levels in mammals. In this reference, the cyclodextrin, specifically hydroxypropyl-beta-cyclodextrin is used as a pharmaceutical delivery agent and not as an active ingredient useful in the reduction of serum cholesterol levels.

U.S. Pat. No. 4,877,778 discusses the administration of doses of 2-hydroxypropyl-beta-cyclodextrin at levels of up to 0.5 gm/kg per day. The cyclodextrin is used as a carrier to remove excess lipophiles from the system, specifically as set forth in the example, reduction of high vitamin A levels. With respect to serum cholesterol levels, the '778 patent suggests that the reduction of serum cholesterol levels achieved in the example is due to the system recognizing an overabundance of cholesterol and the serum cholesterol being subsequently “down-regulated. Such down-regulation is a known biologic phenomenon.” The '778 patent goes on to indicate that it is “the natural cholesterol carrying system which predominates and it is the new homeostasis which must be responsible for the observed drop in serum cholesterol.” Hence, the '778 patent does not suggest that the cyclodextrin is usable as a mechanism to bind bile acids to decrease reabsorption in the lower intestines and is merely cumulative of the prior art which illustrates the use of cyclodextin as a particular pharmaceutical carrier to treat certain disorders.

Publications, patents and patent applications are referred to throughout this disclosure. All references cited herein are hereby incorporated by reference.

All percentages and ratios are calculated by weight unless otherwise indicated. All percentages and ratios are calculated based on the total composition unless otherwise stated.

Thus, there is a need to provide a nutritional food product that is nutritionally balanced and complete as well as one that generates a health benefit, particularly one related to the reduction of harmful cholesterol levels in the blood, without the various side effects or challenges proposed in some of the past solutions.

### SUMMARY OF THE PRESENT INVENTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

The present invention is directed at avoiding the unpleasant task of having to take or ingest pharmaceutical tablets or tolerate dietary supplements or the taste associated with some fortified or modified food products or cereal. The present invention is directed at providing a balanced and complete set of macro and micronutrients in accordance with the U.S. recommended daily values (“USRDV” or “DV”) as well as additional health related benefits. The food product of the present invention achieves a lowering of cholesterol levels through either the unique combination of soluble dietary fiber and certain starches such as cyclodextrins, namely beta-cyclodextrin or increasing the level of cyclodextrins in the product itself.

It has also been surprisingly discovered that by modifying the soluble fiber through hydrolysis, the hypocholesterolemic properties of the fiber can be improved. Such treatment does not adversely impact the taste or texture of the food product, when such treated fiber is included in the product.

The present invention provides a well-balanced and nutritionally complete food product that is palatable and has a good mouth feel and texture so that consumers of the product are not limited solely to those having elevated cholesterol levels, i.e. those total cholesterol levels above 200 mg or those with LDL levels above 130 mg. Consumers with lower cholesterol levels, whether total cholesterol or LDL cholesterol levels, can maintain their average or “good range” cholesterol levels and it is believed, do not experience any adverse effect such as a further lowering of these levels.

In a preferred embodiment of the present invention a food product that has an enhanced cholesterol lowering or triglyceride lowering properties. The food product, comprises at least a first component, which is a starch that has been selected from the group of cyclodextrins. The food product includes at least a second component, such that the starch and the second component provide an increased affinity for binding bile acids.

In an additional embodiment of the present invention a grain based food product is described as having enhanced hypocholesterolemic properties. The food product includes at least a first component which is a starch selected from the group consisting of beta-cyclodextrin, alpha-cyclodextrin and gamma-cyclodextrin. The food product further includes at least a second component that is a sterol based component. The food product is provided with at least a third component that is a dietary fiber. The combination of the first, second and third components have an increased affinity for binding bile acids.

In a still further preferred embodiment of the present invention a method of communicating a beneficial effect of a food product having improved hypocholesterolemic activity is described and includes the steps of initially manufacturing a food product. The food product having at least first, second and third components. Next packaging the food product, then preparing indicia concerning the beneficial effect of the food product and finally releasing the indicia to consumers in at least one of a number of pre-selected formats to increase consumer awareness of the beneficial effect of the food product in maintaining healthy cholesterol levels.

The present invention is intended to be used as a cereal, including ready to eat (RTE) cereals and cereal bars, dairy products such as yogurt, ice cream and other frozen novelties, bakery products such as dough or refrigerated dough, fruit snacks and other snack products. In addition, the present invention is contemplated for use in baking mixes, such as cake, brownie and other desert mixes, ready to eat meals, such as meals that contain pasta and the like.

### BRIEF DESCRIPTION OF THE DRAWINGS

These, as well as other objects and advantages of this invention, will be more completely understood and appreciated by referring to the following more detailed description of the presently preferred exemplary embodiments of the invention in conjunction with the accompanying drawing, of which:

FIG. 1 provides a graphical representation of the results of feeding a food product of the present invention to laboratory animals.

### DETAILED DESCRIPTION OF THE INVENTION

Inclusion of cyclodextrins in ready to eat (RTE) cereals, mixes, doughs, grain based foods and other food products in an amount between 0.1% to 25% by weight, preferably from 2 to 6%, can increase the amount of bile acid binding activity occurring in the gut and thereby reduce total serum cholesterol levels. Cyclodextrin is a product of enzymatic conversion or degradation of starch in which a cyclic ring of sugars is created containing between 5 to 1,000,000 glucose units and more typically between 6 to 8 glucose units. A principal source of cyclodextrins is maize starch. However, cyclodextrins may be derived from a wide variety of plant starches. As indicated above, the cyclodextrins have a hydrophobic core that can bind choloesterol or bile acids and allow these molecules to be excreted from the system in the stool.

Enzymatic degradation or treatment of the starch is done by cyclodextrin glucosyltransferase (EC 2.4.1.19) or other enzymes, which results in a cyclic ring of sugar. The cyclodextrin is resistant to digestion but is susceptible to fermentation by bacteria in the cecum or foregut of the organism. The hydrophobic core serves as binding sites for bile acid and steroids, namely cholesterol. The bond formed between the cyclodextrin and the bile acids and cholesterol is sufficiently strong so as to enable the material to pass through the system without being reabsorbed through the intestines.

The preferred starch of the present invention are cyclodextrins, preferably, beta-cyclodextrin although alpha and gamma-cyclodextrin may also be used in the present invention. As indicated previously, cyclodextrins comprise a doughnut shaped or cyclical structure composed of a number of alpha-D-glucose units (typically 6-8) having a hydrophilic exterior and a hydrophobic interior. Cyclodextrins are generally water soluble, free flowing crystalline powers that are substantially if not completely odorless and white in color. Heretofore, starches such as cyclodextrin were not employed or known for their hypocholesterolemic activity and have been used principally for the encapsulation of insoluble compounds to enhance stability, reduce volatility and alter solubility. Such prior uses of cyclodextrins have been limited to carriers for flavors, therapeutic agents and to remove fatty acids and cholesterol from animal fats

The soluble fiber component of the present invention can be derived from a wide variety of grains and are composed of polysaccharides having a variety of structures. Fiber is generally resistant to human digestive enzymes, except for colonic microflora present in the lower intestines, and are known for their water and ion-binding capacity. The total dietary fiber content as used in the present invention is in the range of about 1 to 20%, preferably around 8 to 14% with a protein content of at least 8 to 9%.

There are a number of sources of dietary fiber suitable for use with the present invention and which provide synergistic functionality with cyclodextrins. These include but are not limited to psyllium, oats and barley.

Psyllium, as used in the present invention, is a known mucilaginous material derived from seeds from the plants of the Plantago genus, Plantago ovata, found in sub-tropical areas. The seeds are dark and shiny and have something of a concave shape to the exterior. Psyllium has been regularly used as a laxative to promote regular bowel function. Psyllium seed may be used in ground, dehusked or in whole form and represents a source of soluble dietary fiber. However, psyllium can have a coarse or rough texture making ingestion occasionally difficult, if the fiber component is not processed in a manner making it readily useable in a consumer food product.

Oat flour as used in the present invention is essentially heat-treated oat groats (hulled, crushed oats) or rolled oats that are ground on a hammer mill or other smooth rolls. There is no separation of the components during the processing of the flour.

Oat bran used in the present invention is produced by grinding clean oat groats or rolled oats and separating the resulting flour by suitable means, such as sieving, into fractions such that the oat bran fraction is not more then 50% of the original starting material. The separated fraction should have at least 5.5% of beta glucan (dry weight basis), and a total dietary fiber content of at least 16% (dry weight basis), so that at least one third of the total dietary fiber is soluble fiber.

Barley, as used in the present invention, is processed in a manner that resembles oats as set forth above, in that it consists of cleaning, hulling, sieving and then grinding. Waxy hulless barley has a higher dietary fiber content than most other sources of fiber and can range from 14 to 20% of the dry weight and have a beta glucan content of around 8 to 10%.

Beta glucans, particularly grain beta glucans (oats and barley), are a known source of dietary fiber and have been included in food products that are used in weight control (beta glucans used as fat substitutes) and as cholesterol lowering additives. The beta glucans that are used in this invention can be naturally occurring or be chemically or enzymatically modified by altering the specific linkages. In addition physical modification of the beta glucans may be achieved by shearing. Beta glucans are obtained from milled cereal grains such as oats and barley (waxy, hulless barley being a particularly good source) in a manner discussed above and are then extracted from the milled grains into warm water and then the solids are removed from the solution.

In the present invention, the first component of this food product formula comes from the soluble fiber group identified above, including psyllium, oat flour, oat bran, barley and beta-glucan. The soluble fiber portion of the present invention ranges from 1 to 25 percent by weight of the total suggested serving size of the product or roughly 55 grams of food product per serving. The preferred weight percentage is about 1 to 18 percent. The soluble fiber component in the examples set forth herein comprises about one to three grams per serving with about 1.4 to 1.6 grams per serving being used in the cereal formulas set forth below.

As used herein the term “grain based” food product means that the food product or a food product intermediate is formed at least in part from the combination of one or more grains such as oats, barley, wheat, corn, psyllium and rice.

As used herein the term “dough” or “doughs” means a food intermediate that is typically subjected to one or more additional processing steps, such as baking, cooking, mixing, etc. before a final food product is available for consumption. Dough is composed of a mixture of at least flour and water.

Another component with which it has been discovered that cyclodextrin has a synergistic benefit are sterols. Sterols occur in natural fats and oils, particularly in vegetable oils. Unsaturated vegetable oils and non-animal fat oils, such as soybean oil, wheat germ oil, cottonseed oil, safflower oil, peanut oil, rice oil, canola oil and the like are well known sources of sitosterol, stigmasterol and campesterol as well as various other materials such as higher aliphatic alcohols. Tall oil is also a significant source of sitosterol and campesterol. The sterol of the present invention may be concentrated by any suitable method such as by (1) saponification, extraction and crystallization; (2) distillation or 3) high pressure propane extraction or obtained from a by-product stream such as from an oil deodorization process.

Stanols are the 5 alpha saturated derivatives of plant sterols and may be derived from similar sources and methods as described above and concentrated. In the present invention, the second component of this food product formula comes from the sterol/stanol group identified above, including beta sitosterol, stigmasterol, campesterol, ergosterol, beta sitostanol, campestanol, stigmastanol and fatty acid derivatives thereof and/or a combination of one or more of the above. The sterol/stanol portion of the present invention ranges from 1 to 25 percent by weight of the total suggested serving size of the product or roughly 55 grams of food product per serving. The preferred weight percentage is about 1 to 18 percent.

The present invention is now illustrated in greater detail by way of the following examples, but it should be understood that the present invention is not to be construed as being limited thereto.

### EXAMPLE 1

In an exemplary embodiment of the present invention, the nutritionally complete food product, in this case a ready to eat (RTE) cereal, may include the following micro and macronutrients in connection with an amount of beta cyclodextrin and a moderate level of dietary fiber. In this example, about 1-3 gms of beta-cyclodexrin is used. The serving size is approximately 55 grams. The ratio of beta-cyclodextrin to dietary fiber ranges from about 1 to 1 to about 1 to 3.5, with other ratios or combinations possible, such as 1 to 1.5, 1.5 to 2, 2 to 1 and 2 to 1.5.

| | Recommended Daily Value (“DV”) |
|---|---|
| Total Fat | 5% |
| Saturated Fat | 3% |
| Cholesterol | 0% |
| Sodium | 10% |
| Potassium | 5% |
| Total Carbohydrate | 14% |
| Dietary Fiber | 17% |
| Protein | 10% |
| Vitamin A | 10% |
| Vitamin C | 70% |
| Calcium | 0% |
| Iron | 80% |
| Vitamin D | 10% |
| Vitamin E | 100% |
| Thiamin | 100% |
| Riboflavin | 80% |
| Niacin | 80% |
| Vitamin B6 | 80% |
| Folic Acid | 100% |
| Vitamin B12 | 100% |
| Phosphorus | 15% |
| Magnesium | 10% |
| Zinc | 80% |
| Copper | 4% |

This example is based on a 2,000 calorie diet and other food products such as cereal bars, fruit snacks, diary and bakery products, baking mixes and ready to eat meals may contain additional vitamins, nutrients and or minerals as well as potentially varying amounts of the macro and micro nutrients set forth in the instant example.

### EXAMPLE 2

In the second example the nutritionally complete food product, in this case a ready to eat (RTE) cereal, may include the following micro and macronutrients in connection with a high beta-cyclodextrin based formula. In this example, about 2-10 gms of beta-cyclodextrin is used. The serving size is approximately 55 grams.

| | Recommended Daily Value (“DV”) |
|---|---|
| Total Fat | 4% |
| Saturated Fat | 3% |
| Cholesterol | 0% |
| Sodium | 10% |
| Potassium | 5% |
| Total Carbohydrate | 26% |
| Dietary Fiber | 5% |
| Protein | 10% |
| Vitamin A | 10% |
| Vitamin C | 70% |
| Calcium | 0% |
| Iron | 80% |
| Vitamin D | 10% |
| Vitamin E | 100% |
| Thiamin | 100% |
| Riboflavin | 80% |
| Niacin | 80% |
| Vitamin B6 | 80% |
| Folic Acid | 100% |
| Vitamin B12 | 100% |
| Phosphorus | 15% |
| Magnesium | 10% |
| Zinc | 80% |
| Copper | 4% |

This example is based on a 2,000 calorie diet and other food products such as cereal bars, fruit snacks, diary and bakery products baking mixes and ready to eat meals may contain additional vitamins, nutrients and or minerals as well as potentially varying amounts of the macro and micro nutrients set forth in the instant example.

The RTE cereals of the preceding examples are prepared in a conventional manner. This exemplary RTE cereal is in the form of flakes that are created by preparing a cooked cereal dough through known methods and then forming the cooked cereal dough into pellets that have a desired moisture content. The pellets are then formed into wet flakes by passing the pellets through chilled roller and then subsequently toasting or heating the wet cereal flakes. The toasting causes a final drying of the wet flakes, resulting in slightly expanded and crisp RTE cereal flakes. The flakes are then screened for size uniformity. The final flake cereal attributes of appearance, flavor, texture, inter alia, are all affected by the selection and practice of the steps employed in their methods of preparation. For example, to provide flake cereals having a desired appearance feature of grain bits appearing on the flakes, one approach is to topically apply the grain bits onto the surface of the flake as part of a coating that is applied after toasting.

While the foregoing examples are directed to the manufacture of flake cereals, it is readily apparent, that the manufacturing method can be modified to produce puffed or extruded cereals as well in which the dough after forming is either fed through an extruder to create the desired shape or in the alternative is forced through a “gun” to generate puffed cereals.

Beta-cyclodextrin as used in the foregoing examples was a “standard grade” and obtained from Cerestar, USA, Inc. of Hammond, Ind. and is marketed under the brand name C★CAVITRON 8200.

While the foregoing example 1 includes reference to dietary fiber as a second component, a sterol based component could also be added to the mixture as a third component or alternatively, the sterol based component would be a substitute for the dietary fiber in achieving the beneficial food product of the present invention.

In each of the foregoing examples, and after completing a prescribed dietary regiment, cholesterol levels in test subjects were seen to be reduced by as much as fifty percent over cereal which did not contain either elevated levels of beta-cyclodextrin alone or the combination of beta-cyclodextrin and a soluble fiber, namely beta-glucan.

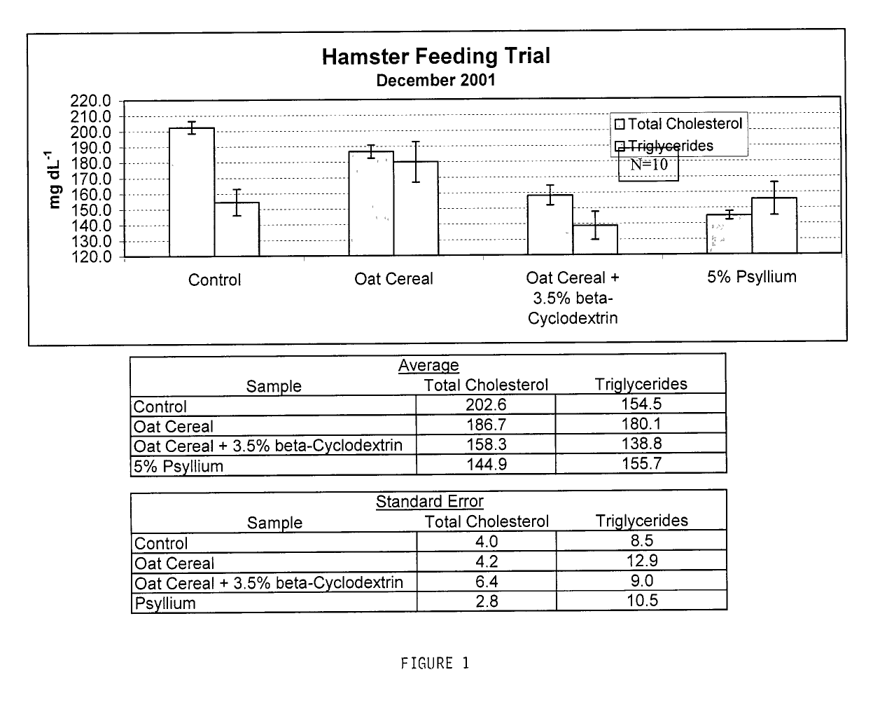

Cereal products prepared in connection with the above-mentioned example 1 were then fed to hamsters and the results compared. A control feed, a feed which is principally an oat cereal (having dietary fiber), an oat cereal having 3.5% concentration of beta-cyclodextrin and dietary fiber and a feed comprising 5% of psyllium. The results of this study are shown in FIG. 1 and provide approximately a 15% reduction in the total cholesterol level as well as the HDL level between the oat cereal and the oat cereal with beta-cyclodextrin.

FIG. 1 also provides that the food product of the present invention resulted in a lowering of triglycerides of about ten percent (10%) when compared with the level of triglycerides in the control formula.

Often and potentially more important that the actual manufacture or delivery of a product is communicating the benefits associated with a particular food product to the consumers. This can be done in a number of ways such as through the preparation of scripted information or indicia that is then released to consumers. The release of such indicia is usually tailored to certain pre-selected or predefined formats and can be done through traditional advertising routes that have at least an audio capability such as radio and television as well printed materials. Printed materials may include the packaging into which the product is placed as well as newspapers, letters, direct mail pieces, magazines and the like.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A food product having enhanced cholesterol-lowering properties, said food product, comprising;
at least a first component, said first component being a starch selected from the group of cyclodextrins;
at least a second component; and
wherein said starch and said second component provide an increased affinity for binding bile acids.

2. A food product as recited in claim 1, wherein said food product is grain based.

3. A food product as recited in claim 1, wherein said cyclodextrin is beta-cyclodextrin.

4. A food product as recited in claim 1, wherein said second component is a dietary fiber.

5. A food product as recited in claim 4, wherein the dietary fiber has been treated to enhance solubility.

6. A food product as recited in claim 4, wherein the dietary fiber is beta glucan.

7. A food product as recited in claim 1, wherein said second component is a sterol based compound.

8. A food product as recited in claim 7, wherein said sterol based component is selected from the group consisting of sitosterol, stigmasterol and campesterol.

9. A food product as recited in claim 1, wherein said food product includes at least a third component.

10. A food product as recited in claim 9, wherein said third component is a micronutrient.

11. A food product as recited in claim 9, wherein said third component is a macronutrient.

12. A food product as recited in claim 9, wherein said third component is a sterol based component.

13. A food product as recited in claim 9, wherein said third component is a dietary fiber.

14. A grain based food product having enhanced hypocholesterolemic properties, said food product comprising;
at least a first component, said first component being starch selected from the group consisting of beta-cyclodextrin, alpha-cyclodextrin and gamma-cyclodextrin;
at least a second component, said second component being a sterol based component;
at least a third component, said third component being a dietary fiber; and
wherein said first, second and third components having an increased affinity for binding bile acids.

15. A grain based food product as recited in claim 14, wherein said sterol based component is selected from the group consisting of sitosterol, stigmasterol and campesterol.

16. A grain based food product as recited in claim 15, wherein said dietary fiber is obtained from the group consisting of oats, barley, psyllium and beta glucan.

17. A method of communicating a beneficial effect of a food product having improved hypocholesterolemic activity comprising the steps of;
manufacturing a food product, said food product having at least first, second and third components;
packaging said food product;
preparing indicia concerning said beneficial effect of said food product; and
releasing said indicia to consumers in at least one of a number of pre-selected formats to increase consumer awareness of said beneficial effect of said food product in maintaining healthy cholesterol levels.

18. A method of communicating as provided in claim 15, wherein some of said pre-selected formats have at least an audio capability.

19. A method of communicating as provided in claim 16, wherein some of said pre-selected formats are printed.

20. A food product having enhanced triglyceride-lowering properties, said food product, comprising;
at least a first component, said first component being a starch selected from a group of cyclodextrins;
at least a second component; and
wherein said starch and said second component provide an increased affinity for binding bile acids.

21. A food product intermediate having enhanced cholesterol lowering properties, comprising;
at least a first component, said first component being a starch selected from a group of cyclodextrins;
wherein said food product intermediate with said at least first component provide an increased affinity for binding bile acids.
